(19) United States
(12) Patent Application Publication
(10) Pub. No.: US 2022/0226807 A1
(43) Pub. Date: Jul. 21, 2022

(54) LOW-VOLUME SYSTEMS FOR SAMPLE IDENTIFICATION

(71) Applicant: LexaGene, Inc., Beverly, MA (US)

(72) Inventors: Gregory A. Dale, Wayland, MA (US); Nick Moses, Wakefield, MA (US); Roman Paz, Watertown, MA (US); Keith Sproat, Truckee, CA (US); Richard L. Miller, Needham, MA (US)

(73) Assignee: LexaGene, Inc., Beverly, MA (US)

(21) Appl. No.: 17/614,045

(22) PCT Filed: May 22, 2020
(86) PCT No.: PCT/US2020/034179

§ 371 Date: Nov. 24, 2021

Related U.S. Application Data
(60) Provisional application No. 62/853,759, filed on May 29, 2019.

(51) Int. Cl. B01L 3/02

(52) U.S. Cl. CPC B01L 3/0293; B01L 2200/16; B01L 3/50853; B01L 2300/1894; B01L 2400/0487; B01L 2300/0838

(57) ABSTRACT

The invention includes low-volume systems for sample identification. The systems are designed to use multi-use consumables including but not limited to fluid containers containing large volumes of fluids. In some embodiments, the low-volume systems identify nucleic acids in samples using polymerase chain reaction (PCR).

### CROSS-REFERENCE TO RELATED APPLICATIONS

This application claims priority to U.S. Provisional Patent Application No. 62/853,759 filed May 29, 2019. The foregoing application is hereby incorporated by reference in its entirety.

### FEDERALLY SPONSORED RESEARCH OR DEVELOPMENT

Not applicable.

### TECHNICAL FIELD

The invention generally relates to low-volume systems for sample identification. The invention more specifically relates to low-volume systems for identifying nucleic acids in a sample.

### BACKGROUND

Sample identification systems generally generate large amounts of waste by employing single-use consumables, such as reaction sites (including but not limited to defined flow paths and flow cells), fluid containers, and sample preparation cartridges. Typically, the large amounts of waste ends up in landfills, where it does not degrade and can leach hazardous substances, or is incinerated and can release hazardous substances. In addition, single-use consumables are a major expense to the purchaser/user of identification systems and over time may cost more than the rest of the identification system, including identification instruments.

Thus, there is a need for low-volume systems for sample identification that overcome the aforementioned problems and limitations.

### SUMMARY

The invention reduces single-use consumable waste by using multi-use consumables. In particular, the invention allows for the reuse of fluid containers (making them multi-use consumables) by using fluid containers containing large volumes of fluids and reducing the volume of fluidics in the overall identification system. The identification systems disclosed herein are low-volume systems and, in some embodiments, designed to be used with fluid containers containing large volumes of fluids. Priming and flushing (also referred to as “purging”) the fluidics of the identification system requires large amounts of fluids, so reducing the overall volume of the fluidics significantly reduces the amount of fluids consumed during priming and flushing. In addition, the invention includes reaction sites that can also be reused, which results in additional waste reduction. As a result, the invention aims to reduce the cost per sample test for the purchaser/user of the identification system.

Unless otherwise defined, all terms used herein have the same meaning as commonly understood by a person having ordinary skill in the art to which the invention pertains. All patents, patent applications, publications, and other references mentioned herein and/or listed in the Application Data Sheet are hereby incorporated by reference in their entirety. In case of conflict, the specification will control. When a range of values is provided, the range includes the end values.

The materials, methods, components, features, embodiments, examples, and drawings disclosed herein are illustrative only and not intended to be limiting.

### DESCRIPTION OF DRAWINGS

The invention is best understood from the following detailed description when read in connection with the drawings disclosed herein, with similar elements having the same reference numbers. When a plurality of similar elements is present, a single reference number may be assigned to the plurality of similar elements with a small letter designation referring to at least one specific similar element. When referring to the similar elements collectively or to a non-specific similar element, the small letter designation may be dropped. The various features of the drawings may not be drawn to scale and may be arbitrarily expanded or reduced for clarity. Included in the drawings are the following figures:

FIG. 1 is a diagram depicting a system for loading at least one fluid in accordance with aspects of the invention.

FIG. 2 is a diagram depicting a system for loading at least one fluid in accordance with aspects of the invention.

FIG. 3 is a diagram depicting a system for loading at least one fluid in accordance with aspects of the invention.

FIG. 4 is a diagram depicting a system for loading at least one fluid in accordance with aspects of the invention.

FIG. 5 is a diagram depicting two fluid containers in accordance with aspects of the invention.

FIG. 6 is a diagram depicting a bay and a fluid container loaded into the bay in accordance with aspects of the invention.

FIG. 7 is a schematic representing a low-volume system for sample identification in accordance with aspects of the invention.

FIG. 8 is a schematic representing a low-volume system for sample identification using PCR in accordance with aspects of the invention.

### DEFINITIONS

To facilitate understanding of the invention, a number of terms are defined in alphabetical order herein.

“Fluid container” means a container containing at least one fluid in at least one fluid reservoir. A fluid container includes but is not limited to a vessel, rack, frame, and plate.

“Fluid reservoir” means a defined area of the fluid container that is capable of holding at least one fluid. A fluid reservoir includes but is not limited to a well, tube, tubing, channel, and compartment.

“PCR master mix” means a solution of reagents for a polymerase chain reaction (PCR) reaction. A PCR master mix typically includes polymerase and deoxynucleotides (dNTPs) (and/or similar nucleotides) and typically does not include probes or primers.

“Reagent pack” means a fluid container containing at least one reagent in at least one fluid reservoir. An example of a reagent pack includes but is not limited to a rack with at least one tube wherein the at least one tube contains at least one reagent, a frame with at least one tube wherein the at least one tube contains at least one reagent, a plate with a least one well wherein the at least one well contains at least one reagent, a plate with at least one channel wherein the at least one channel contains at least one reagent, and a vessel with at least one compartment wherein the at least one compartment contains at least one reagent.

### DETAILED DESCRIPTION

The invention minimizes the volumes of the fluidics in the identification system to allow for multi-use consumables, including but not limited to multi-use fluid containers. In certain aspects, the invention minimizes the volumes of the fluidics in the system for loading at least one fluid (also referred to as “loading system”), which is a part of the identification system. The identification system can be the same as or similar to the system disclosed in U.S. Pat. No. 8,298,763 and hereby incorporated by reference in its entirety. The identification system can be used to identify a variety of samples for a variety of purposes. For a first example, the identification system can be used to identify samples from animals for veterinarian purposes. For a second example, the identification system can be used to identify samples from fruits and vegetables for food-safety purposes. For a third example, the identification system can be used to identify samples from water for water quality monitoring purposes. For a fourth example, the identification system can be used to identify samples from humans for biosafety purposes.

In the loading system, at least one needle (that is used to interface with the at least one fluid in the fluid container) is directly attached to or in close proximity to a manifold, thereby eliminating or reducing the tubing and/or microfluidic channels between the at least one needle and the manifold. In addition, a valve is directly attached to or in close proximity to the manifold, thereby eliminating or reducing the tubing and/or microfluidic channels between the valve and the manifold. In some embodiments, the bay (that receives the fluid container) is raised to establish fluidic connectivity between the at least one needle and at least one fluid in a fluid container. In other embodiments, the manifold is lowered to a fluid container to establish fluidic connectivity between the at least one needle and at least one fluid in a fluid container. In other embodiments, the bay is raised and the manifold is lowered to establish fluidic connectivity between the at least one needle and at least one fluid in a fluid container.

The invention also contemplates fluid containers with large volumes of at least one fluid. The large volumes of the at least one fluid in combination with the minimized volumes of the fluidics in the identification system allows the at least one fluid to be used multiple times, meaning the at least one fluid is pumped to the reaction site multiple times. The number of times that the at least one fluid can be pumped to the reaction site includes but not limited to 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 200, 300, 400, 500, or more times. In some embodiments, the fluid container is a reagent pack containing at least one fluid reservoir containing a PCR master mix and at least one fluid reservoir containing at least one probe and at least one set of primers, and the reagent pack is used at least 10 times, meaning that the reagents are pumped from the reagent pack to the reaction site at least 10 times.

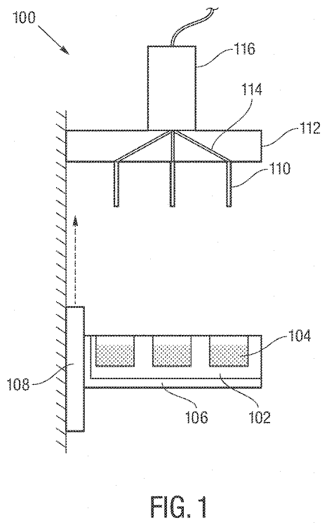

FIG. 1 is a diagram depicting a system for loading at least one fluid in accordance with aspects of the invention. In this diagram, the loading system 100 includes a fluid container 102, three fluids 104 (in three fluid reservoirs), a bay 106, an elevator 108, three needles 110, a manifold 112, three microfluidic channels 114, and a valve 116. The three fluids 104 are in the fluid container 102. Three fluids were chosen in this diagram for illustrative purposes only and any amount of fluids could be in the fluid container including but not limited to 1, 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 200, or more fluids. Each fluid can be in any type of fluid reservoir. The fluid reservoir can be a permanent part of a fluid container and therefore not removable from the fluid container. For example, a non-removable fluid reservoir can be a well or a permanently attached tube in a fluid container. Alternatively, a fluid reservoir can be a non-permanent part of a fluid container and therefore removable from the fluid container. For example, a removable fluid reservoir can be a tube that is removably attached to the fluid container by, for example, snapping (inserting) the tube into a hole in the fluid container. An example of a suitable tube includes but is not limited to the Micrewtube® graduated tube (Simport Scientific, Beloeil, Quebec, Canada).

In FIG. 1, the fluid container 102 is loaded into (including onto) the bay 106, which is configured to receive the fluid container, and the bay is connected to the elevator 108. A fluid container can be loaded into a bay in a number of methods including but not limited to top-loaded, front-loaded, back-loaded, side-loaded, bottom-loaded, and a combination thereof. Examples of loading methods include but are not limited to placing (moving substantially vertically) a fluid container into a bay, sliding (moving substantially horizontally) a fluid container into a bay, and a combination thereof. A fluid container can also be loaded in a two-step process such as first placing a fluid container into a bay and then sliding the fluid container and bay into (including onto) a loading system so that the fluid container and bay engage with an elevator. In this diagram, the elevator 108 is attached to the bay 106 so that the elevator can raise the bay. An elevator and a bay can be permanently or removably attached. For example, an elevator can be welded (permanently attached) to a bay or magnetically connected to a bay (removably attached). In other embodiments, a bay and an elevator are the same unit. A bay can be raised by a number of methods including but not limited to a piston to push the bay, an electric motor that raises the bay (in some embodiments, an elevator connected to the bay) along a rod, chain, track, and/or cable, and the manual or automated insertion of a wedge under the bay to push the bay. The dashed arrow represents the raising of the bay. In this diagram, the elevator 108 raises the bay to the three needles 110, which are in fluidic connectivity with the manifold 112, so that the three needles 110 interface with the three fluids 104 to establish fluidic connectivity between the three needles 110 and the three fluids 104. Fluidic connectivity can be established by the three needles 110 fully or partially entering the three fluids 104. Three needles were chosen in this diagram for illustrative purposes only but any amount of needles could be in fluidic connectivity with a manifold including but not limited to 1, 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 200, or more needles. The manifold 112 can be made of any rigid material including but not limited to metal or plastic. In a preferred embodiment, the manifold is made of a thermoplastic.

In FIG. 1, the number of needles and fluids are the same (three) for illustration purposes only, and the number of needles and fluids can be different. For example, a loading system can have a manifold in fluidic connectivity with four needles and a fluid container with 16 fluids. In addition, in some embodiments the needles and/or the fluids are independently moveable. In this diagram, the three needles 110 are attached directly to the manifold 112, however, one or more needles can be in fluidic connectivity with a manifold by tubing and/or additional microfluidic channels. The manifold 112 contains three microfluidic channels 114 with each microfluidic channel having an inlet connected to a needle 110 and an outlet in fluidic connectivity with the valve 116. In preferred embodiments, the loading system contains the same number of needles as there are fluids in the fluid container.

In FIG. 1, the valve 116 is attached directly to (mounted on) the manifold 112 however, a valve can be in fluidic connectivity with a manifold by tubing and/or additional microfluidic channels. Each microfluidic channel can have any shape and dimension but, in preferred embodiments, each microfluidic channel has a volume between 5 μL and 25 μL, preferably a volume of no more than 20 μL. The valve 116 can be any type of microfluidic valve and in preferred embodiments is attached directly to (mounted on) the manifold 112. In this diagram, the three fluids 104 in the fluid container 102 are pumped through the three needles 110 through the three microfluidic channels 114 and into the valve 116. The valve 116 selectively allows at least one fluid at a time to travel out of the valve 116 and towards a reaction site (not depicted).

A loading system is typically part of an identification system (other components of identification system not depicted). The loading system is designed to minimize the volume of the fluidics, along with the other components of the identification system, to allow for reusable consumables such as fluid containers (including reagent packs) and sample preparation cartridges. In FIG. 1, the three needles 110, the manifold 112, and the valve 116 are all directly attached to (mounted on) one another to minimize the volume of the fluidics of the loading system. A person skilled in the art will understand that tubing and/or additional microfluidic channels can be used anywhere in the loading system, but it will add to the volumes of the loading system and thereby reduce the value of the loading system as part of a low-volume identification system.

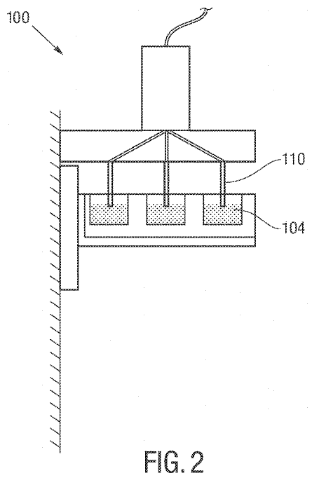

FIG. 2 is a diagram depicting a system for loading at least one fluid in accordance with aspects of the invention. FIG. 2 depicts the loading system 100 after the bay is raised and the three needles 110 are in fluidic connectivity with the three fluids 104.

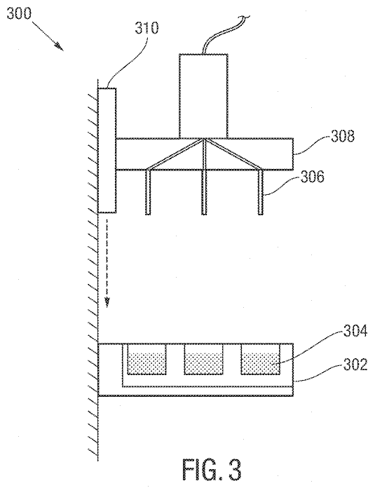

FIG. 3 is a diagram depicting a system for loading at least one fluid in accordance with aspects of the invention. A loading system 300 in FIG. 3 is similar to the loading system 100 in FIG. 1. However, unlike the loading system 100, a manifold 308 of the loading system 300 is lowered to the fluid container 302 to establish fluidic connectivity between three needles 306 and three fluids 304 (in three fluid reservoirs). The manifold 308 is attached to an elevator 310 so the elevator 310 can lower the manifold. An elevator and a manifold can be permanently or removably attached. For example, an elevator can be welded (permanently attached) to a manifold or magnetically connected to a manifold (removably attached). In other embodiments, a manifold and an elevator are the same unit. A manifold can be lowered by a number of methods including but not limited to a piston to push the manifold, an electric motor that lowers the manifold (in some embodiments, an elevator connected to the manifold) along a rod, chain, track, and/or cable, and the manual or automated insertion of a wedge above the manifold to push the manifold.

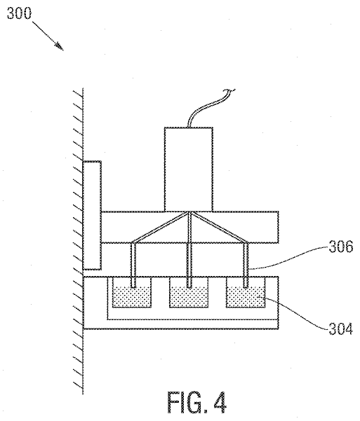

FIG. 4 is a diagram depicting a system for loading at least one fluid in accordance with aspects of the invention. FIG. 4 depicts the loading system 300 after the manifold is lowered and the three needles 306 are in fluidic connectivity with the three fluids 304.

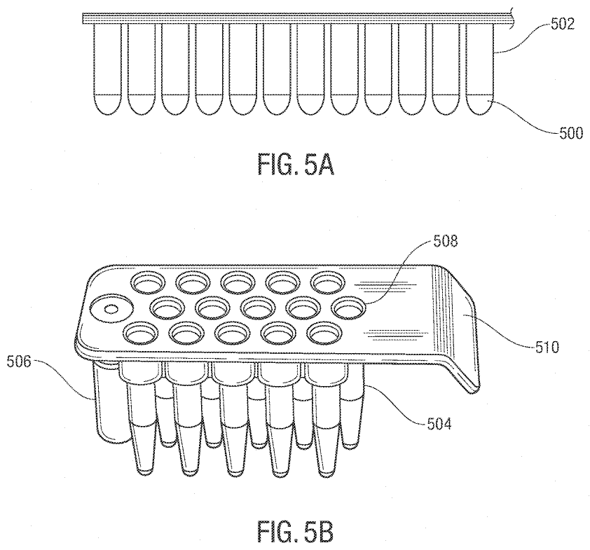

FIG. 5 is a diagram depicting two fluid containers in accordance with aspects of the invention. FIG. 5A is a diagram depicting a fluid container containing fluids 500 in fluid reservoirs 502. The fluid reservoirs 502 are arranged in a single row. Each fluid 500 can be any fluid including but not limited to reagents. In this diagram, the fluid reservoirs 502 are tubes and the tubes can be either fixed or removable. In some embodiments, a fluid container and fluid reservoirs are the same unit. A fluid container can be made of any rigid material including but not limited to metal and plastic and can be created by a variety of methods including molding, extrusion, and additive manufacturing (3D printing). 3D printing a fluid container with holes and then inserting standard size (off-the-shelf) tubes into the holes (to create a finished fluid container) reduces the cost of manufacturing and therefore reduces the cost to the purchaser/user. Each fluid in a fluid container can have any volume that can be contained in a given fluid reservoir of the fluid container including but not limited to 10 microliters (μL), 20 μL, 30 μL, 40 μL, 50 μL, 60 μL, 70 μL, 80 μL, 90 μL, 100 μL, 150 μL, 200 μL, 250 μL, 300 μL, 350 μL, 400 μL, 450 μL, 500 μL, 600 μL, 700 μL, 800 μL, 900 μL, 1000 μL, 1500 μL, 2000 μL, 2500 μL, 3000 μL, 3500 μL, 4000 μL, 4500 μL, 5000 μL, 6000 μL, 7000 μL, 8000 μL, 9000 μL, 10000 μL, or more μL. In preferred embodiments, each fluid has a volume between 200 μL and 1000 μL.

FIG. 5B is a diagram depicting a fluid container containing 16 fluid reservoirs in the form of two types of tubes (in multiple rows), which are 15 smaller-volume tubes 504 and a larger-volume tube 506. In some embodiments, the larger-volume tube 506 contains a PCR master mix and the 15 smaller-volume tubes 504 contain other PCR reagents such as aqueous PCR assays. Each of the 15 smaller-volume tubes can contain the same or different aqueous PCR assays. In other embodiments, the larger-volume tube 506 contains a PCR master mix and the 15 smaller-volume tubes 504 contain a combination of PCR reagents such as aqueous PCR assays and cleaning fluids used to periodically clean and/or sterilize the identification system. In these embodiments, the PCR reagents and the cleaning fluids are in different tubes.

Each tube contains a membrane covering its opening, which is punctured by a needle when the fluid container (loaded in a bay) is raised to the needle (which is in fluidic connectivity with a manifold). The membrane can be made of rubber, silicone, or a similar elastomer and can be pre-slit to facilitate puncturing and also resealing when a needle is removed. The membrane helps to prevent the evaporation of the fluid in the tube and also helps to prevent contamination. A fluid container can also include at least one additional layer of material on top of some or all of the fluid reservoirs. The at least one additional layer includes but is not limited to at least one additional membrane, a metal film (such as an aluminum or aluminum mylar film), and a printed label that covers all or part of the top of the fluid container and identifies the fluid(s) in the fluid reservoir(s). The fluid container depicted in FIG. 5B includes a handle 510 to facilitate the loading and unloading of the fluid container. A fluid container can have any shape, such as square or circular, and fluid reservoirs can be in a regular pattern, such as rows and/or circles, or in an irregular pattern.

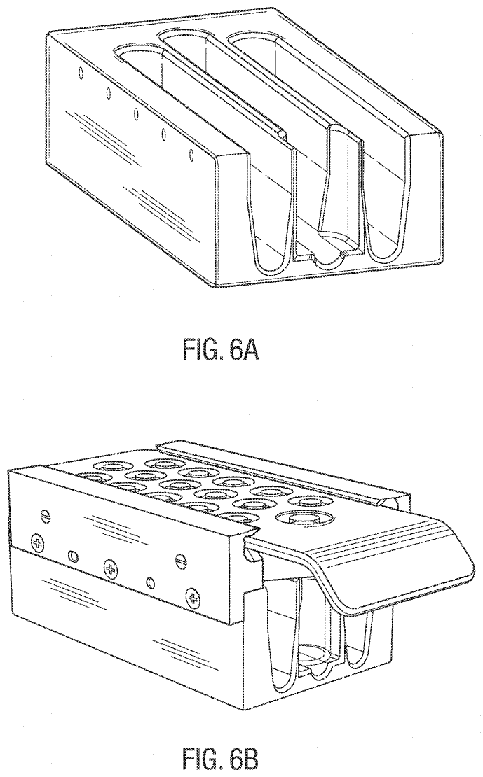

FIG. 6 is a diagram depicting a bay and a fluid container loaded into the bay in accordance with aspects of the invention. FIG. 6A is a diagram depicting a bay configured to receive a fluid container. A bay can be designed to cool part or all of a fluid container by passive and/or active cooling and can be made of any rigid material, preferably a metal or metal alloy. Cooling can be by any method including but not limited to convection, conduction, and radiation. In preferred embodiments, the fluid container is cooled by a Peltier cooling device to a temperature between 4 degrees Celsius (° C.) and 8° C. Cooling all or part of a fluid container helps to extend the life of the fluid(s) in a fluid container and prevent evaporation. In this embodiment, the bay receives the fluid container by sliding to front-load the fluid container. FIG. 6B is a diagram depicting the bay of FIG. 6A loaded with a fluid container. A bay and a fluid container can be designed to reversibly lock together.

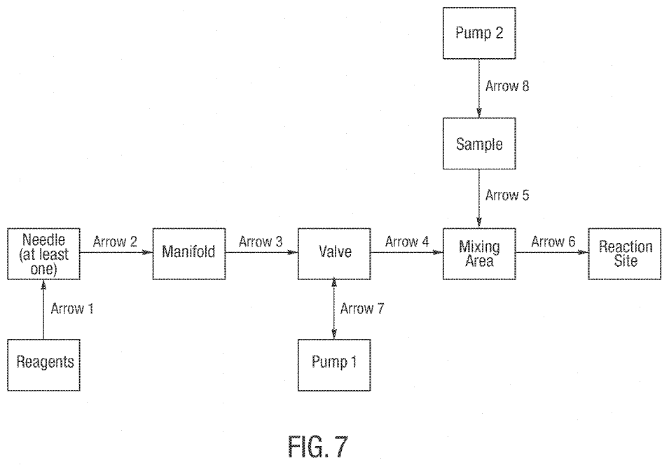

FIG. 7 is a schematic representing a low-volume system for sample identification in accordance with aspects of the invention. In this schematic, the boxes represent components of the system and the arrows represent fluidic connectivity between components and the direction of fluid flow. For example, arrow 4 between the valve and the mixing area represents that the valve and mixing area are in fluidic connectivity and that fluid flows from the valve to the mixing area. The arrows can also represent a means of fluidic connectivity between components including but not limited to tubing and microfluidic channels. For example, arrow 4 between the valve and the mixing area can also represent at least one tubing and/or microfluidic channel between the valve and the mixing area. All of the components of FIG. 7 are in fluidic connectivity with one another. The reagents, which are in a fluid container, flow to the at least one needle, then to the manifold, then to the valve, then to the mixing area (where the reagents are mixed with sample) and then to the reaction site. In this schematic, pump 1, which is in bidirectional fluidic connectivity with the valve, and pump 2, which is in unidirectional fluidic connectivity with the sample, cause fluid flow. In a preferred embodiment, arrow 1 represents fluidic connectivity only and is not a means of connectivity (for example, is not at least one tubing and/or microfluidic channel) because the at least one needle is fully or partially in the reagents (directly connected to the reagents) and therefore is the means of fluidic connectivity between the reagents and the manifold, arrow 2 and arrow 3 each represent fluidic connectivity only and are not a means of connectivity because the at least one needle and valve are both attached directly to (mounted on) the manifold (and the at least one microfluidic channel in the manifold is the means of fluidic connectivity between the at least one needle and the valve), arrow 4 is at least one tubing, arrow 5 is at least one tubing, arrow 6 is at least one tubing, arrow 7 is at least one tubing, and arrow 8 is at least one tubing. To minimize the volume of the fluidics in the schematic, and thereby create a low-volume system, the volume for each of the means of connectivity (arrows) should be minimized. In preferred embodiments, the volume of arrow 1 is 0 μL (because the at least one needle is fully or partially in the reagents and is the means of fluidic connectivity between the reagents and the manifold), the volume of each needle is between 0.1 μL and 1 μL, the volume of arrow 2 is 0 μL, the volume of each microfluidic channel in the manifold is between 1 μL and 20 μL, the volume of arrow 3 is 0 μL, the volume of each tubing of arrow 4 is between 2 μL and 100 μL, the volume of each tubing of arrow 5 is between 5 μL and 300 μL, the volume of each tubing of arrow 6 is between 2 μL and 25 μL, the volume of each tubing of arrow 7 is between 5 μL and 100 μL, and the volume of each tubing of arrow 8 is between 5 μL and 100 μL. In preferred embodiments, the volume of the fluidics of the entire low-volume system for sample identification (including components and the fluidic connectivity between the components) is not greater than 375 μL.

In FIG. 7, pump 1 and pump 2 can each be any microfluidic pump including but not limited to a peristaltic pump, syringe pump, pressure pump, and positive displacement pump. An example of a microfluidic pump is a PSD/4 Precision Syringe Drive (Hamilton Company, Reno, Nev., USA). The valve can be any microfluidic valve, for example the TitanHT™ rotary shear valve (IDEX Corporation, Lake Forest, Ill., USA). The mixing area can be any microfluidic mixing area and can use passive and/or active mixing. For example, the mixing area can be a serpentine shaped microfluidic channel (passive mixing) or a magnetic micro-stirrer (active mixing). The sample may contain at least one nucleic acid of interest (also referred to as a “target nucleic acid”), and, in some embodiments, the at least one nucleic acid of interest is identified by fluorescence-based PCR. The reaction site is the where the reaction occurs. Types of reaction sites include but are not limited to defined flow paths, including but not limited to tubing and microfluidic channels, and flow cells. Defined flow paths are disclosed in PCT International Patent Application No. PCT/US20/29199, which is hereby incorporated by reference in its entirety.

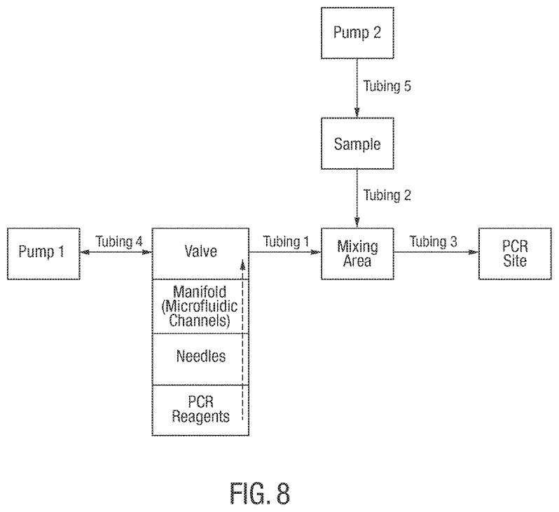

FIG. 8 is a schematic representing a low-volume system for sample identification using PCR in accordance with aspects of the invention. In this schematic, the reagents are PCR reagents and are in tubes. At least one tube is a PCR master mix and at least one tube is an aqueous PCR assay including but not limited to at least one probe and at least one set of primers. The probe is a fluorescent probe such as a TaqMan® probe (Thermo Fisher Scientific, Waltham, Mass., USA), which is a probe linked to a fluorophore and a quencher. The PCR reagents are pumped to the mixing area via the needles, the microfluidic channels in the manifold, the valve, and tubing 1 where they are mixed with the sample, which may contain at least one target nucleic acid, to create reaction mixtures. The sample is pumped to the mixing area via tubing 2. The reaction mixtures are then pumped to the PCR reaction site via tubing 3 where fluorescence-based PCR occurs. In fluorescence-based PCR, at least one fluorescent probe fluoresces under certain conditions, and the fluorescence is captured (imaged) and used to determine if the at least one target nucleic acid was present in the sample. If the at least one target nucleic acid was present in the sample, then it may be possible to identify the sample. Pump 1 is in bidirectional fluidic connectivity with the low-volume system for sample identification via tubing 4, and pump 2 is in unidirectional fluidic connectivity with the low-volume system for sample identification via tubing 5.

In FIG. 8, the needles are mounted on a side of the manifold and the valve is mounted on the same or another side of the manifold. Each needle is connected to the valve via a microfluidic channel in the manifold. The dashed arrow represents the flow of PCR reagents to the valve. In a preferred embodiment, the PCR reagents are in a reagent pack comprising 16 fluid reservoirs in the form of 16 tubes. 16 needles are mounted on a side of the manifold and the needles are in fluidic communication with the PCR reagents in the 16 tubes.

What is claimed is:

1. A system for loading at least one fluid, comprising:
a bay;
a fluid container comprising at least one fluid; and
a manifold in fluidic connectivity with at least one needle;
wherein the fluid container is loaded in the bay and wherein fluidic connectivity is established between the at least one fluid and the at least one needle by at least one of (i) raising the fluid container and (ii) lowering the manifold.

2. The system for loading at least one fluid of claim 1, wherein the bay is configured to cool at least part of the fluid container.

3. The system for loading at least one fluid of claim 2, wherein the fluid container is front-loaded into the bay.

4. The system for loading at least one fluid of claim 1, wherein the at least one fluid comprises at least one reagent.

5. The system for loading at least one fluid of claim 4, wherein the at least one reagent is polymerase chain reaction (PCR) reagents.

6. The system for loading at least one fluid of claim 5, wherein the polymerase chain reaction (PCR) reagents are in an aqueous solution with a volume of at least 200 microliters (μL).

7. The system for loading at least one fluid of claim 1, wherein the manifold comprises at least one microfluidic channel comprising an inlet and an outlet, wherein the at least one needle is in fluidic connectivity with the inlet, and wherein the volume of the at least one microfluidic channel is not greater than 20 microliters (μL).

8. A multi-use reagent system, comprising:
a fluid container, comprising at least one reagent;
a manifold in fluidic connectivity with at least one needle;
at least one pump; and
a reaction site;
wherein the at least one needle is in fluidic connectivity with the at least one reagent, wherein the manifold is in fluidic connectivity with the at least one pump and the reaction site, and wherein the at least one reagent is pumped to the reaction site by the at least one pump at least 10 times.

9. The multi-use reagent system of claim 8, wherein the reaction site is a defined flow path.

10. The multi-use reagent system of claim 9, wherein the defined flow path is at least one of a tubing and a microfluidic channel.

11. The multi-use reagent system of claim 8, wherein the at least one reagent is polymerase chain reaction (PCR) reagents and the reaction site is a polymerase chain reaction (PCR) site.

12. The multi-use reagent system of claim 10, wherein the polymerase chain reaction (PCR) reagents are an aqueous solution with a volume of at least 200 microliters (μL).

13. The multi-use reagent system of claim 8, wherein the manifold comprises at least one microfluidic channel comprising an inlet and an outlet, wherein the at least one needle is in fluidic connectivity with the inlet, and wherein the volume of the at least one microfluidic channel is not greater than 20 microliters (μL).
